(19) United States
(12) Patent Application Publication
(10) Pub. No.: US 2017/0356915 A1
(43) Pub. Date: Dec. 14, 2017

(54) BLADDER CANCER PROGNOSIS

(71) Applicant: The University of Birmingham, Birmingham, West Midlands (GB)

(72) Inventors: Douglas George WARD, Birmingham, West Midlands (GB); Richard Trevor BRYAN, Birmingham, West Midlands (GB)

(21) Appl. No.: 15/529,937

(22) PCT Filed: Nov. 27, 2015
(86) PCT No.: PCT/GB2015/053630

§ 371 Date: May 25, 2017

Related U.S. Application Data
(60) Provisional application No. 62/085,433, filed on Nov. 28, 2014.

(51) Int. Cl. G01N 33/574; C12Q 1/68; G01N 33/68

(52) U.S. Cl. CPC G01N 33/57407; G01N 33/6893; C12Q 1/6886; C12Q 2600/118

(57) ABSTRACT

The present invention concerns the use of protein biomarkers for use in facilitating in the prognosis and/or treatment regime of bladder cancer. In particular, the invention relates to the use of shed protein fragments, such as fragments of Epithelial cell adhesion molecule (EpCAM) and/or epidermal growth factor receptor (EGFR) detected in a sample of urine, as biomarkers for use in facilitating the prognosis and/or treatment regime of urothelial bladder cancer.

### FIELD OF THE INVENTION

The present invention concerns the use of protein biomarkers for use in facilitating in the prognosis and/or treatment regime of bladder cancer. In particular the invention relates to the use of shed protein fragments, such as extracellular fragments, as biomarkers for use in facilitating in the prognosis and/or treatment regime of urothelial bladder cancer.

### BACKGROUND OF THE INVENTION

Urothelial bladder cancer (UBC) is the seventh commonest cancer in Western societies (Burger et al, 2013). At presentation 75-85% will be non-muscle-invasive tumours (NMIBC, stages pTa/pT1/pTis), with the remainder being muscle-invasive (MIBC, stages pT2-4) (Kaufman et al, 2009). Progression to or presentation with MIBC represents the critical step in the disease course, necessitating radical therapies, including chemoradiotherapy or neoadjuvant chemotherapy followed by radical cystectomy (Witjes et al, 2014). Despite such approaches, outcomes have changed little for three decades (Kaplan et al). Reliably identifying patients with the most aggressive UBCs early (both NMIBC and MIBC) and expediting aggressive therapeutic strategies could go some way to improving outcomes (Bryan et al, 2014a), yet this remains a significant challenge despite sophisticated clinico-pathological algorithms (Babjuk et al, 2011; Witjes et al, 2014).

Since urine directly contacts UBCs it is an attractive biospecimen for developing non-invasive tests to detect and characterise bladder tumours. Challenges in developing such tests include UBC heterogeneity, such that different tumours may release different biomarkers (thus necessitating multimarker tests) and early-stage and low-grade tumours may only release very small amounts of such markers, potentially impairing test sensitivity. Additionally, markers must be highly tumour-specific so that haematuria and other non-malignant conditions do not generate false positives. In the search for better urinary biomarkers genomic, proteomic and metabolomic approaches have all yielded promising results (Huang et al, 2011; Kandimalla et al, 2013; Orenes-Piñero et al, 2007).

It is amongst the objects of the present invention to provide one or more urine markers which may be of use in providing a prognosis and/or therapeutic strategy for subjects with UBC.

### SUMMARY OF THE INVENTION

The present invention is based in part on observations by the present inventors that certain membrane proteins which are present on the surface of urothelial bladder cancer (UBC) cells release a fragment of the membrane protein into urine and that the level of such released protein fragment(s) may be correlated with a survival prognosis of the subject.

In a first aspect there is provided a method for facilitating in the prognosis of a subject having urothelial bladder cancer (UBC), the method comprising:

detecting in a sample of urine, a level of one or more fragments of Epithelial cell adhesion molecule (EpCAM) and/or epidermal growth factor receptor (EGFR) which are shed by UBC cells into the urine;

wherein the subject is determined to have a poor prognosis when a level of each or all of said one or more fragments of EpCAM and/or EGFR in the sample of urine is/are elevated and

wherein the subject is determined to have a good prognosis when a level of all of said one or more fragments of EpCAM and/or EGFR in the sample of urine is/are not elevated

It is to be appreciated that a poor prognosis may be associated with an elevated level of only one of said fragments of EpCAM and/or EGFR, but for a subject to have a good prognosis there should not be an elevated level of either EpCAM or EGFR.

The fragments of the present invention are protein fragments of a whole membrane protein which are released or shed into a subject's urine. Typically, such protein fragments comprise at least a portion of an extracellular region of the identified proteins. The fragments may be characterised by way of being soluble, as opposed to the native whole proteins which are membrane bound and would not be soluble.

In an embodiment of the present invention, levels of both EpCAM and EGFR are detected. In such an embodiment, elevated levels of one or optionally both EpCAM and EGFR may be associated with a poor prognosis for cancer survival. In another embodiment, only the level of EpCAM or EGFR is detected. In one embodiment, only the level of EGFR is detected. In some embodiments, the level of EGFR and optionally the level of EpCAM is detected.

In one embodiment a level in a sample of urine of one or more further protein fragments may also be detected, such as hepatocyte growth factor activator inhibitor type 1 (HAI-1) and/or midkine (MDK).

It must be appreciated that embodiments of the present invention are concerned with prognosis rather than diagnosis. Prognosis may be considered as a prediction of a probable course and outcome of a disease, as opposed to diagnosis which is concerned the identification of disease.

Advantageously, the present invention is based on the detection of protein fragment(s), especially soluble protein fragments, in urine samples. Urine is easy to collect and can be provided easily by a subject themselves, without the necessary need of a health care worker, such as may be required for obtaining blood samples. The urine sample or urine sample container may be pretreated as necessary for storage or preservation, by dilution in an appropriate buffer solution or concentrated, if desired. Any of a number of standard aqueous buffer solutions, employing one of a variety of buffers, such as phosphate, Tris, or the like, at physiological pH can be used. The urine sample can in certain circumstances be stored for use prior to use in the methods or assays as disclosed herein. Such storage can be at +4° C. or frozen, for example at −20° C. or −80° C.

As used herein, the term “subject” is preferably a mammal. The mammal can be a human, non-human primate, mouse, rat, dog, cat, horse, or cow, but is not limited to these examples.

As used herein, the term “poor prognosis” refers to a subject that is unlikely to survive 5 years, 4 years, 3 years, 2 years or 1 year from testing. The term “unlikely” means that on average this applies to greater than 75% of subjects.

As used herein, the term “good prognosis” means that a subject is likely to survive for at least 5 years 4 years, 3 years, 2 years or 1 year from testing. The term “likely” means that on average this applies to greater than 75% of subjects.

An elevated level of a chosen protein is understood to mean a level which is two standard deviations (2SD) or more above a normal or a reference value, which is typically a mean of a normal reference range. A non-elevated level is within a normal reference range (as determined from subjects without UBC) meanplus 2SD. Such a normal/reference value may be made/normalised with reference to a level of another metabolite which may also ideally be present in urine, such as a level of creatinine or urea for example. This allows for variations in hydration to be accounted for. In accordance with an embodiment of the present invention an elevated level of EGFR may be understood to be >630 pg EFGR/mg creatinine and consequently a non-elevated level of EGER may be understood to be <630 pg EGFR/mg creatinine. In accordance with an embodiment of the present invention an elevated level of EpCAM may be understood to be >24 pg EpCAM/mg creatinine and consequently a non-elevated level of EpCAM may be understood to be <24 pg EpCAM/mg creatinine.

For any other protein fragments which may be included in addition to the aforementioned EpCAM and EGFR, the skilled addressee can easily determine an elevated and non-elevated value in accordance with the above criteria. For example, a elevated/non-elevated level threshold for MDK is 9.1 ng/mg creatinine and for HAI-1 is 1.2ng/mg creatinine based on mean +2SD in non-cancer controls.

It may not be necessary to further treat the urine sample, but if desired, the protein fragments may be isolated or separated from other proteins which may be present in the sample of urine. This may be achieved through, for example centrifugation and/or chromatographic techniques known to the skilled addressee. For example, the sample of urine may be subjected to a centrifugation or ultracentrifugation process in order to pellet non-soluble material and/or proteins and leave the protein fragments of interest in solution. The solution after centrifugation can then be analysed in order to detect the level of a particular protein fragment. The protein fragments of interest in the present invention may also be isolated/separated by way of adsorption chromatography techniques, such as affinity (such as immunoaffinity) and/or ion-exchange techniques known to the skilled addressee. Such chromatography techniques may bind or retard the protein fragments of interest, as opposed to other proteinaceous material not of interest, or vice versa.

It may also be possible and preferred, when required, to isolate/separate the protein fragments of interest from other proteinaceous material, based on size. For example, dialysis, filtration, ultrafiltration and/or size exclusion chromatography techniques are known to the skilled addressee and may be used to isolate/separate the protein fragments of interest from other proteinaceous material. Filtration/ultrafiltration may be particularly preferred as it is relatively easy and quick to carry out.

Additionally, electophoresis techniques, such as denaturing and non-denaturing electrophoresis, isolelectric focusing, and two-dimensional electrophoresis techniques may be employed to isolate/separate the proteins of interest, levels of which can be subsequently be detected.

The particular protein fragments may be can be detected or isolated using techniques, including but not limited to immunohistochemistry, Western blot analysis, immunoblotting, ELISA, immunoprecipitation, lateral flow immunoassay, radioimmunoassay and levels of the protein fragments quantified accordingly. One example of a suitable immunoassay is the xMAP® technology as provided by Luminex.

Antibodies can also be raised against the protein fragments by methods known to those skilled in the art. Antibodies are readily raised in animals such as rabbits or mice by immunization with the gene product, or a fragment thereof. Immunized mice are particularly useful for providing sources of B cells for the manufacture of hybridomas, which in turn are cultured to produce large quantities of monoclonal antibodies. While both polyclonal and monoclonal antibodies can be used in the methods described herein, it is preferred that a monoclonal antibody is used where conditions require increased specificity for a particular protein fragment. Antibody manufacture methods are described, for example, in Harlow et al., 1988. The antibodies that recognize the protein fragments of the present invention may be any antibody variant, antibody derivative, bispecific molecule, human antibody, humanized antibody, monoclonal antibody, human monoclonal, and variants and antigen-binding fragments thereof. Conventional methods for immunohistochemistry are described in Harlow and Lane, 1988 and Ausbel et al, 1987.

The antibodies may also be labeled with a radio, chemical, chemiluminescent or any other suitable label.

The protein fragments of the present invention may be detected and levels determined using mass spectrometry techniques, such as a matrix assisted laser desorption/ionisation mass spectrometric (MALDI-MS) technique or LC-MS/MS and selected reaction monitoring.

In one embodiment, a sample may be obtained at more than one time point from the same individual that is to be tested as described herein. In such instances, the various samples can provide a measure of the efficacy of treatment, or the development of disease.

The present invention may be of use in determining what type of medical intervention may be required for a particular subject, or to ascertain whether or not a particular therapy is appropriate based on the levels of EpCAM and/or EGFR detected. For example, if a subject is identified in accordance with the present invention as having a poor prognosis, then this may direct a medical practitioner to adopt a rapid and/or aggressive strategy for therapy on the subject, for example early cystectomy for poor prognosis subjects with non-invasive disease or cystectomy instead of radiotherapy for muscle-invasive disease.

Also, dependent on the prognosis evaluation, it may be decided that anti-EpCAM and/or anti-EGFR therapies may be appropriate therapies to administer to a subject.

Thus, in a further aspect, there is provided a method of facilitating in the determination of treatment to a subject with UBC, the method comprising:

detecting in a sample of urine, a level of one or more fragments of Epithelial cell adhesion molecule (EpCAM) and/or epidermal growth factor receptor (EGFR) which are shed by UBC cells into the urine;

wherein the subject is determined to have a poor prognosis when a level of each or all of said one or more fragments of EpCAM and/or EGFR in the sample of urine is/are elevated; and

wherein the subject is determined to have a good prognosis when a level of all of said one or more fragments of EpCAM and/or EGFR in the sample of urine is/are not elevated; and selecting a therapy for the subject dependent upon the subject being identified as having a poor or good prognosis.

In one embodiment, the method comprises detecting a level of one or more fragments of epidermal growth factor receptor (EGFR) only. In some embodiments, the method comprises detecting a level of one or more fragments of epidermal growth factor receptor (EGFR), and optionally, Epithelial cell adhesion molecule (EpCAM).

The method may further include the step of administering/conducting the selected therapy. For subjects with a poor prognosis, the therapy may be cystectomy and/or anti-EpCAM/anti-EGFR therapy.

There is thus also provided a method of administering/conducting a therapy to a subject with UBC, the method comprising:

detecting in a sample of urine, a level of one or more fragments of Epithelial cell adhesion molecule (EpCAM) and/or epidermal growth factor receptor (EGFR) which are shed by UBC cells into the urine;

wherein the subject is determined to have a poor prognosis when a level of each or all of said one or more fragments of EpCAM and/or EGFR in the sample of urine is/are elevated; and

wherein the subject is determined to have a good prognosis when a level of all of said one or more fragments of EpCAM and/or EGFR in the sample of urine is/are not elevated;

selecting and administering/conducting a therapy to the subject dependent upon the subject being identified as having a poor or good prognosis.

Thus, there is also provided a method of treating a subject with UBC, the method comprising:

detecting in a sample of urine, a level of one or more fragments of Epithelial cell adhesion molecule (EpCAM) and/or epidermal growth factor receptor (EGFR) which are shed by UBC cells into the urine;

wherein the subject is determined to have a poor prognosis when a level of each or all of said one or more fragments of EpCAM and/or EGFR in the sample of urine is/are elevated; and

administering an effective amount of therapy to the subject, wherein the therapy is administered to a subject that has elevated levels of EpCAM and/or EGFR.

For subjects with a poor prognosis, the therapy may be cystectomy and/or anti-EpCAM/anti-EGFR therapy.

In some embodiments, the therapy is a compound. The compound includes, but is not limited to mitomycin C or BCG induction

The table below indicates possible treatments regimes for subjects identified as displaying a good or poor prognosis based on the detection of levels of EGFR and/or EpCAM in accordance with the present invention, in combination with determination of bladder cancer disease category determination following surgical procedures.

| | Biomarker-determined | Biomarker-determined |
|---|---|---|
| Bladder cancer | good prognosis | poor prognosis |
| disease category | Management | Management |
| Low-risk NMIBC* | Post-TURBT cystoscopic | Post-TURBT cystoscopic |
| | surveillance for up to | surveillance beyond 1 |
| | 1 year | year and up to 10 years |
| Intermediate-risk | Post-TURBT adjuvant | Post-TURBT adjuvant |
| NMIBC* | intravesical mitomycin | intravesical BCG |
| | C x 6 doses | induction and up to |
| | | one year maintenance |
| High-risk NMIBC* | Post-TURBT adjuvant | Radical cystectomy |
| | intravesical BCG | |
| | induction and up to | |
| | three years maintenance | |

*Risk determination as carried out in accordance with Babjuk M, Burger M, Zigeuner R, Shariat S F, van Rhijn B W, Comperat E, Sylvester R J, Kaasinen E, Bohle A, Palou R J, Roupret M. EAU guidelines on non-muscle-invasive urothelial carcinoma of the bladder: update 2013. Eur Urol 2013; 64: 639-653. TURBT = Trans-urethral resection of bladder tumour; BCGinduction = 6 weekly treatments of BCG into the bladder; BCG maintenance = 3 treatments weekly every 3 months.

In accordance with the invention, assay systems are also provided. The assay systems include a measurement device that measures a level of one or more fragments of Epithelial cell adhesion molecule (EpCAM) and/or epidermal growth factor receptor (EGFR) in a urine sample, in order to provide data in relation to the level of EpCAM and/or EGFR fragments in urine. The system also includes a data transformation device that acquires the EpCAM and/or EGFR fragment level(s) data from the measurement device and performs data transformation to calculate whether or not the level determined is elevated or not, in accordance with the present invention.

In other embodiments, the assay system also includes a user interface output device to output data to a user. In other preferred embodiments, the assay system also includes a database of treatment information, wherein the device identifies treatment information in the database for the level of EpCAM and/or EGFR fragment(s) determined and outputs the treatment information to the user interface output device.

In a further aspect there is provided a kit for use in the present methods. The kit can comprise at least one antibody, or probe which is/are capable of specifically binding to EpCAM and/or EGFR protein fragment(s), and may be labelled for example with a chemical, fluorescent or luminescent label and optionally instructions for use in a method as described herein. One example would be an xMAP beads coated with the aforementioned antibodies.

### DETAILED DESCRIPTION OF THE INVENTION

The present invention will now be further described by way of example and with reference to the attached tables and figures which are not to be construed as limiting.

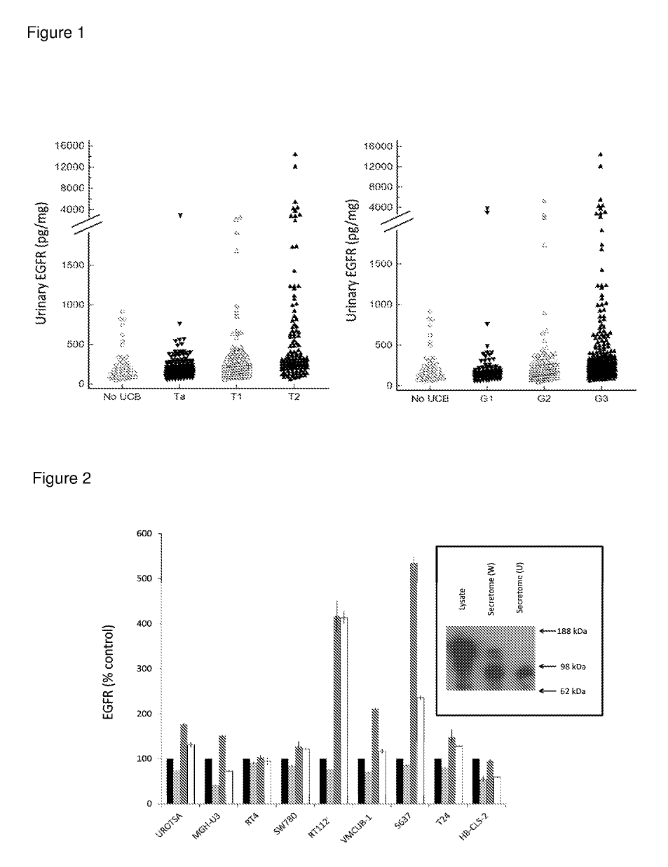

FIG. 1 shows the urinary EGFR in UBC patients. The left hand panel shows urinary EGFR data for patients stratified according to stage and the right hand panel according to grade.

FIG. 2 shows the effects of ultracentrifugation and PMA stimulation on cell line secretomes. Cells were incubated overnight in serum free media±200 nM PMA and EGFR measured in the conditioned media by ELISA pre-ultracentrifugation (black bars=control, dark grey bars=PMA) or post-ultracentrifugation (light grey bars=controls, white bars=PMA). Data are presented relative to the control for each cell line. The insert shows an EGFR Western blot of HB-CLS-2 cell lysate and secretome (W=whole, U=ultracentrifuged).

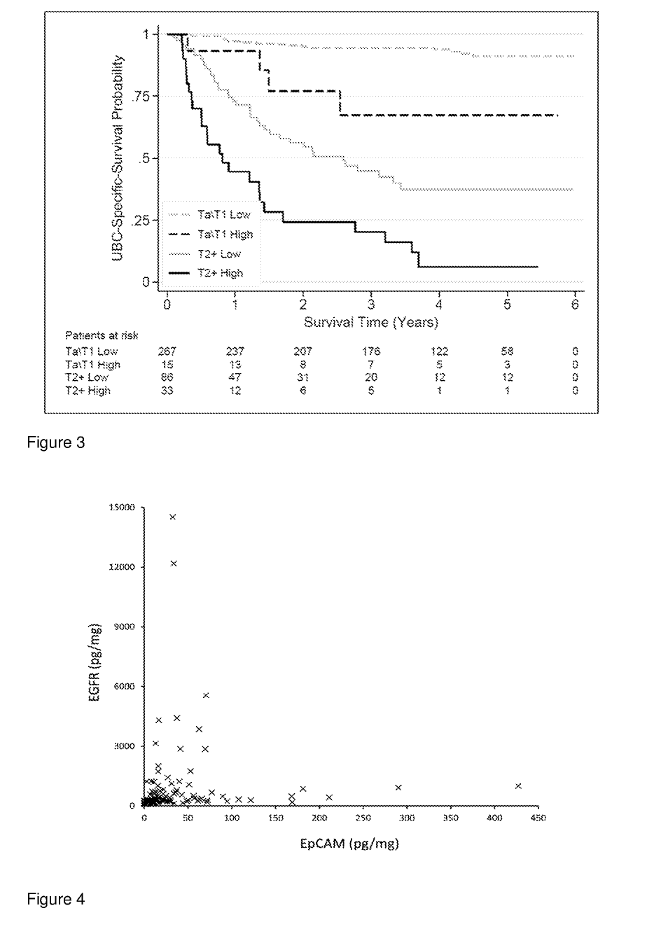

FIG. 3 shows the prognostic value of urinary EGFR. Kaplan-Meier curves are shown for UBC-specific survival with patients stratified according to low/high urinary EGFR (>630 pg/mg creatinine).

FIG. 4 shows the relationship between urinary EGFR and EpCAM in UBC: the creatinine-normalised concentrations of EGFR and EpCAM in the urine of 113 patients with MIBC.

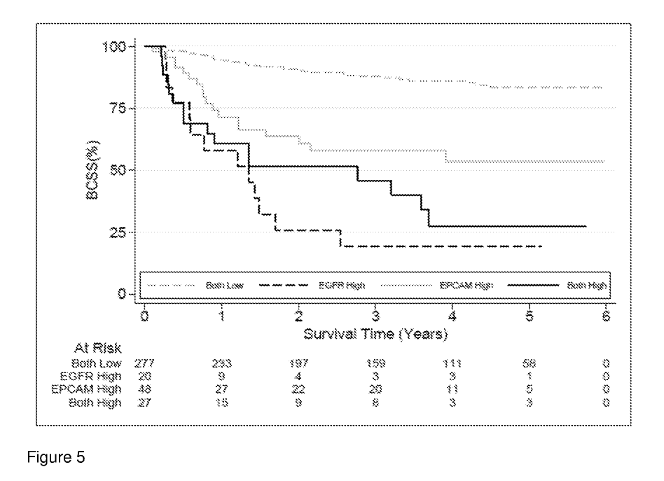

FIG. 5 shows the prognostic value of urinary EGFR and EpCAM. Kaplan-Meier curves are shown for bladder cancer-specific survival with patients stratified according to low/high urinary EGFR and EpCAM.

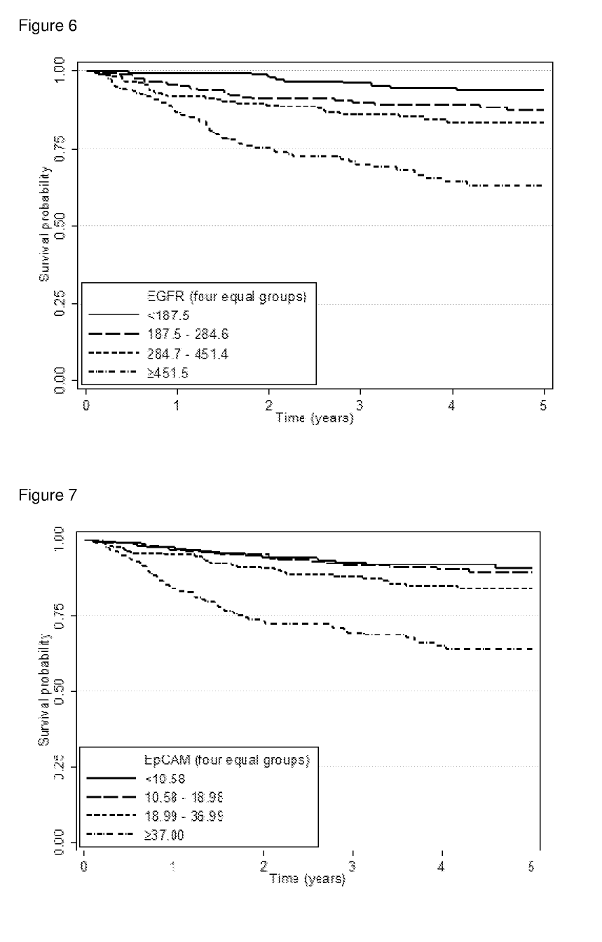

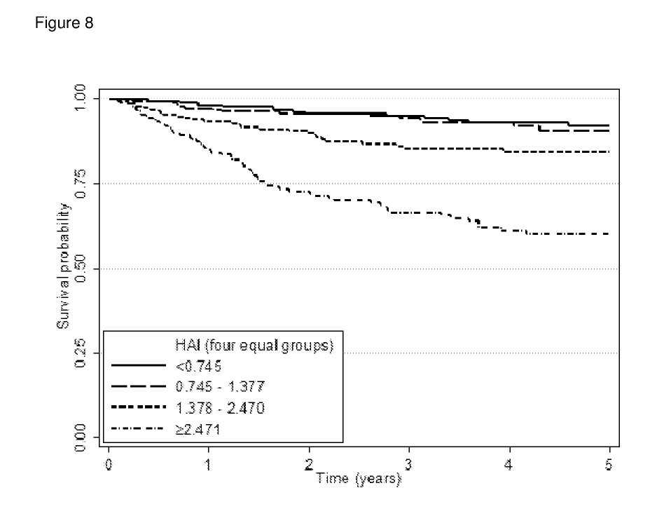

Since the original EGFR study detailed herein and with reference to FIGS. 1-5, the patient cohort has been expanded to include 911 bladder cancer patients. Kaplan-Meier survival curves for bladder cancer specific survival in the expanded cohort stratified into quartiles dependent on EGFR, EpCAM and HAI-1 concentration are shown in FIGS. 6-8.

Materials and Samples

The cell lines used in this study were selected on the basis that they exhibit diverse mutation profiles and may capture some of the heterogeneity of UBC (Supplementary Data, Table 1). Urine samples were prospectively collected for biomarker research between 2006 and 2009 as part of the Bladder Cancer Prognosis Programme (BCPP, ethics approval 06/MRE04/65) (Zeegers et al, 2010). Patients were enrolled on the basis of initial cystoscopic findings suggestive of primary UBC. All UBC patients were newly-diagnosed, had not received treatment for UBC prior to urine collection, and were subsequently treated according to current standard practice. Inclusion and exclusion criteria are detailed elsewhere (Zeegers et al, 2010). Samples were placed on ice, centrifuged at 2000 rpm for 10 minutes within 8 hours of collection, and supernatant stored at −80° C. Since patient recruitment occurred prior to histopathological confirmation of UBC, a proportion of patients were ultimately diagnosed with non-malignant conditions and these serve as non-cancer ‘controls’. All patients were followed-up to July 2014 with causes of death notified to the BCPP study office.

Proteomic Analysis of Secretomes

Secretomes were harvested and analysed by shotgun proteomics as described previously (Shimwell et al, 2013), except that secretomes were collected for each cell line with or without 200 nM phorbol 12-myristate 13-acetate (PMA). Proteins were digested with trypsin and stable isotope labelled (dimethylation with formaldehyde/deuterated formaldehyde) to allow relative protein concentrations±PMA to be calculated.

Cells were grown to confluence (apart from RT4 cells which grow as islands). Adherent cells were then washed 4 times with serum-free media and incubated overnight in serum-free media±200 nM phorbol 12-myristate 13-acetate (PMA). The conditioned media were centrifuged at 1500×g for 20 minutes prior to filter-aided tryptic digestion. Proteins were reduced with 20 mM DTT in 8M Urea, 1% CHAPS, 100 mM TEAB (1 hour at room temperature), alkylated with 40 mM iodoacetamide and then concentrated in 30 kDa centrifugal filters. Following 4 washes with 100 mM triethylammonium bicarbonate, sequencing grade trypsin was added to the filters (1:50 trypsin:protein) and incubated at 37° C. overnight. The peptides were centrifuged through the filters and their primary amines dimethylated by incubating with 25 mM sodium cyanoborohydride and 0.2% formaldehyde (control secretome) or 0.2% deuterated formaldehyde (+PMA secretome) for 30 min at room temperature. Following quenching with 100mM ammonium bicarbonate the samples were fractionated by mixed-mode HPLC and analysed by LC-MS/MS and the data searched against Swissprot human and bovine sequence databases using MASCOT. Multiple datasets were combined using Proteinscape 3 (Bruker Daltronics) and filtered at a 1% protein false discovery rate. Light and heavy dimethylation of N-termini and lysine side-chains were included as variable modifications and relative quantitation was performed using WARP-LC software (Bruker Daltonics).

EGFR ELISA

EGFR was measured with a sandwich ELISA utilising goat polyclonal antibodies against the extracellular domain of EGFR (R&D Systems, Cat no. DYC1854-5). Between 2 and 100 μl of secretome or 50 μl of urine plus 50 μl of 1% BSA in PBST was used per well. Urinary EGFR is expressed as pg EGFR per mg creatinine.

Human Proteome Atlas Analysis

Data was downloaded from http://www.proteinatlas.org and a list of 54 proteins overexpressed in UBC drawn up on the basis that they exhibited high or medium staining in at least 4 cases (out of 11 or 12 cases depending on the protein) and low or undetectable staining in normal urothelium.

Statistical Analyses

Urinary EGFR levels are presented as medians and statistical significance was calculated using Mann-Whitney tests. UBC-specific survival was defined as the time from registration into the BCPP study to date of death from UBC. Patients were censored at the date last known to be alive or date of UBC-related death. Univariable Cox proportional hazards models were employed (alpha 0.1) to identify factors to be included in a Cox multivariable model; significance was set at 0.05. Analysis was done in Stata 12.1 (StataCorp, College Station, Tex., USA).

Results

Proteomic Analysis of Secretomes

In total, 2104 proteins were identified in the conditioned media of one or more cell lines (Table 2). To select candidate biomarkers we considered proteins released by at least 3 of the 8 cancer cell lines and also reported to be overexpressed in UBC in the Human Protein Atlas. This generated a shortlist of 5 candidates: EGFR, G6PD, PRDX6, LYPD3 and FBLN1. EGFR was unique in that it was the only one of these 5 proteins that was identified in the cancer cell line secretomes and not in the UROtsa secretome. Tryptic peptides from EGFR were detected in 5 of the 8 UBC cell lines with a tendency for more peptides (suggestive of a higher EGFR protein concentration) in the cell lines derived from higher grade tumours (Table 2). The levels of EGFR in the secretomes were subsequently measured by ELISA and found to reflect the number of peptides identified. EGFR secretome levels also reflected levels in the corresponding cell lysates as determined by Western blotting (data not shown).

Urinary EGFR as a Diagnostic Marker

EGFR was measured in the urine of 436 UBC patients and 60 non-cancer controls. The data are summarised in Table 3. The median urinary EGFR concentration was 153 pg/mg creatinine in the non-cancer controls and was not significantly increased in patients with pTa UBC. There were significantly higher levels of EGFR in the urine of patients with pT1 or pT2+ UBC (224 and 317pg/mg creatinine, respectively). The data for individual patients stratified according to stage and grade is shown in FIG. 1. High levels of urinary EGFR were observed in a proportion, but not all, patients with high grade and high stage UBC. Defining the upper limit of the normal reference range as the mean +2SD gave a threshold of 630 pg EGFR/mg creatinine, and the percentage of positive cases is 7, 4, 13, and 27 in non-cancer controls, pTa, pT1 and pT2+ UBC, respectively.

Characterisation of ‘Soluble’ EGFR

Peptides from the extracellular domain, but not the intracellular domain, of EGFR were identified in secretomes from MGH-U3, RT112 and 5637 cell lines. VM-CUB-1 and HB-CLS-2 secretomes contained the highest levels of EGFR, and peptides from both the intracellular and extracellular domains were identified, however, both spectral counting and peptide intensity suggested a higher concentration of the extracellular domain than the intracellular domain i.e. the extracellular domain of EGFR is shed into the conditioned media and membrane-bound EGFR is also present. To test this hypothesis we ultracentrifuged the secretomes at 136,000×g for 90 minutes to pellet membranes and membrane-bound proteins and measured the EGFR remaining in the supernatants. On average, across the secretomes, 73% of the EGFR remained in the supernatant confirming that most of the EGFR is a soluble rather than membrane-bound form (FIG. 2). Western blotting of the proteins released by HB-CLS-2 cells indicated that the predominant form of EGFR in the secretome has a molecular weight of approximately 100 kDa. A small amount of full-length EGFR (approximately 170 kDa) is also present but is removed by ultracentrifugation (FIG. 2). The ratio of heavy to light EGFR peptide intensities in the mass spectra suggested that EGFR is released constitutively but can be further stimulated by PMA, and this was confirmed by ELISA: an increase in the amount of EGFR released (both total and soluble forms) was seen with most of the cell lines and was as high as 4-5-fold in some cases (FIG. 2).

Ultracentrifugation was also used to test whether urinary EGFR was shed extracellular domain or membrane-bound EGFR: the EGFR concentrations of 12 urine samples were measured pre- and post-ultracentrifugation. On average, 93.5% (SD 7.8%) remained in the supernatant, indicating that the predominant form of EGFR in urine is soluble extracellular domain.

Urinary EGFR as a Prognostic Marker

Univariable analyses included age (years), multiple tumours (1 vs 2+), grade (1, 2 vs 3), size of largest tumour (p<3 cm vs >3 cm), CIS (present vs absent), stage (pTa or pT1 vs pT2+), sex (male vs female) and urinary EGFR (normal vs elevated). Grade, stage, age, tumour size and CIS all reached statistically significance and were included in the multivariable analysis. In univariable analysis EGFR was found to be highly significant (HR 6.9 90% CI 4.7, 10.1, p<0.001) and this can be seen in Kaplan-Meier curves for UBC-specific survival in both NMIBC and MIBC cases (FIG. 3). Multivariable analysis showed that elevated urinary EGFR (624 pg EGFR per mg creatinine) is prognostic (HR 2.9 95% CI 1.1, 4.6: P<0.001) in addition to grade and stage for UBC-specific survival (Table 4).

Combination of Urinary EpCAM and EGFR for Prognostication

We have previously reported that the urinary concentration of the extracellular domain of EpCAM is associated with a shorter UBC-specific survival time (Bryan et al, 2014b the entire contents of which are herein incorporated by reference, and which is appended hereto by way of reference). There is little correlation between the secretome levels of EpCAM and EGFR across UBC cell lines (Table 2), suggesting that using these proteins might act as prognostic markers independently of one another. Similarly there is little correlation between the urinary concentrations of EGFR and EpCAM in UBC patients: many patients have high levels of either EpCAM or EGFR, with 33 MIBC patients positive for EGFR, 45 positive for EpCAM and 59 positive for one or both proteins (52% of the MIBC patients) (FIG. 4). Multivariable modeling including both biomarkers was conducted. Grade and stage both remained highly prognostic (p=0.011, p<0.001 respectively), as did both EGFR (HR=6.69 95% Cl 3.41, 13.12 p<0.001) and EpCAM (HR=2.13 95% Cl 1.19, 3.82 p=0.011) (Table 5). The risk associated with having elevated levels of both biomarkers was found to be greater than those with only EpCAM elevation, but less than those with only elevated EGFR (p=0.001) (FIG. 5).

Discussion

Combining proteomic analysis of UBC cell line secretomes and immunohistochemistry data available in the Human Protein Atlas identified EGFR as a potential urinary biomarker for UBC. We then tested the diagnostic and prognostic utility of urinary EGFR in a cohort of 496 patients with long-term clinical follow-up. Urinary EGFR is elevated in 27% of MIBC cases and 17% of grade 3 UBCs but normal levels are observed in pTa and low-grade disease; thus, overall, urinary EGFR is not a good diagnostic marker for UBC. However, urinary EGFR is a strong independent prognostic indicator of UBC-specific survival. Urinary EGFR could have utility for rapidly identifying patients with the most aggressive disease, and expediting their subsequent investigation and management.

Shedding of EGFR from cancer cell lines (although not UBC cell lines) has been reported to be related to overexpression, to be stimulated by PMA (and hence may be under the control of protein kinase C), and to be blocked by metalloprotease inhibitors (Perez-Torres et al, 2008). Adamczyk reported that pancreatic cancer cell lines release both exosomal (full length, 170 kDa) and ectodomain (110 kDa) forms of EGFR (Adamczyk et al, 2011). The evidence suggests that EGFR ectodomain is shed as the result of proteolytic cleavage and that it is probably cleavage of an alternatively spliced 3 kb transcript of EGFR that generates soluble ectodomain (Wilken et al, 2013). Although this splice variant lacks both the transmembrane and intracellular domains it seems that it remains membrane associated until cleaved (Wilken et al, 2013). Our UBC cell line data is consistent with the body of evidence suggesting that both full-length and ectodomain EGFR are released from cancer cells by a regulated mechanism involving proteolytic cleavage.

Soluble EGFR has been detected in plasma and investigated in several cancers. However, plasma levels appear to be lower in cancer patients than healthy controls, are not related to EGFR expression in tumours and there is little evidence of diagnostic, predictive or prognostic value (Baron et al, 2003; Hudelist et al, 2006; Lemos-González et al, 2007; Müller et al, 2006; Zampino et al, 2008). Although EGF has been measured in the urine of UBC patients (Chow et al, 1997), the only case of EGFR being measured in urine that we have encountered looks at a number of distal tumour sites and not UBC (Witters et al, 1995). Thus, we believe that the current work is the first exploration of soluble urinary EGFR as a biomarker for UBC, with a much clearer relationship between urinary EGFR and UBC than in studies of plasma EGFR in other solid malignancies.

EGFR has been reported to be overexpressed in many epithelial cancers and was reported as a poor prognostic indicator in UBC as early as 1990 (Neal et al, 1190), representing a late event in the progression of UBC (Lipponen & Eskelinen, 1994). More recently, EGFR overexpression has been shown to be a characteristic of basal-like aggressive MIBC, and in mouse models these tumours respond to anti-EGFR therapy (Rebouissou et al, 2014). Several clinical trials using anti-EGFR therapies in combination with chemotherapy or radiotherapy in MIBC are currently underway.

Without wishing to be bound by theory, the urinary levels of EGFR may be due to the level of expression in tumour cells, and/or due to shedding of the EGFR ectodomain. Thus, urinary EGFR could be a facile indicator of a patient's suitability to be treated with anti-EGFR therapy.

Conclusions

We present the first description of the prognostic value of soluble urinary EGFR in UBC, both alone and in combination with soluble urinary EpCAM (Bryan et al, 2014b the entire contents of which are herein incorporated by reference). These ELISA-based tests are easy and reproducible, and thus have significant potential clinical utility and we intend to evaluate this potential prospectively in UBC. Mechanisms of ectodomain shedding may add an extra layer of complexity to the molecular pathology of UBC that may not be uncovered by genomic approaches and appear to unmask potentially important prognostic markers. Better understanding of these phenomena may also reveal new therapeutic targets, targets that are desperately needed for UBC (Bryan et al, 2014a).

TABLE 1
Urothelial cell lines. 5637 and HB-CLS-2 were purchased
from CLS Cell Lines Service GmbH (Eppelheim, Germany). VM-
CUB-1, MGH-U3, RT4, RT112, SW780 and T24 were validated
and selected on the basis that they exhibit a diversity
of mutation profiles and therefore may capture some of the
heterogeneity of UBC. The UROtsa immortalised normal urothelium
cell line was a gift from Alexander Dowell.

| | Grade of | | |
|---|---|---|---|
| Cell Line | tumour | Mutations | Growth conditions |
| UROtsa | N/A | ND | RPMI-1640 + 10% FCS |
| MGH-U3 | 1 | TP53: WT | DMEM + 10% FCS + |
| | | FGFR3: Y375C | 2 mM L-glutamine + 1% |
| | | RAS: WT | non-essential amino acids |
| | | PIK3CA: WT | |
| | | TSC1: WT | |
| | | AKT1: E17K | |
| RT4 | 1 | TP53: WT | McCoy's 5A + 10% |
| | | FGFR3: | FCS + 2 mM L-glutamine |
| | | translocation | |
| | | RAS: WT | |
| | | PIK3CA: WT | |
| | | TSC1: | |
| | | c.1669delC | |
| | | AKT1: WT | |
| SW-780 | 1 | TP53: WT | DMEM + 10% FCS + |
| | | FGFR3: | 2 mM L-glutamine |
| | | translocation | |
| | | RAS: WT | |
| | | PIK3CA: WT | |
| | | TSC1: WT | |
| | | AKT1: WT | |
| RT112 | 2 | TP53: WT | RPMI-1640 + 10% FCS |
| | | FGFR3: | |
| | | translocation | |
| | | RAS: WT | |
| | | PIK3CA: WT | |
| | | TSC1: WT | |
| | | AKT1: WT | |
| VM-CUB-1 | 2 | TP53: R175H | DMEM + 10% FCS + |
| | | FGFR3: WT | 2 mM L-glutamine |
| | | RAS: WT | |
| | | PIK3CA: E545K | |
| | | TSC1: WT | |
| | | AKT1: WT | |
| 5637 | 2 | TP53: R280T | RPMI-1640 + 10% FCS |
| | | RAS: WT | |
| | | PIK3CA: WT | |
| | | TSC1: WT | |
| | | AKT1: WT | |
| T-24 | 3 | TP53: Y126* | DMEM + 10% FCS + |
| | | FGFR3: WT | 2 mM L-glutamine |
| | | RAS: | |
| | | HRAS p.G12V | |
| | | PIK3CA: WT | |
| | | TSC1: WT | |
| | | AKT1: WT | |
| HB-CLS-2 | 3 | ND | RPMI-1640 + 10% FCS |

TABLE 2
UBC cell line secretome Analysis. The total number of unique peptides
identified by LC-MS/MS in each secretome and the resulting protein
identifications are shown alongside the number of EGFR peptides
identified. The EGFR and EpCAM concentrations (determined by ELISA
using the same set of secretomes for both proteins) are also shown
(mean (SD) for triplicate measurements).

| | No. | No. | No. | | |
|---|---|---|---|---|---|
| | human | human | EGFR | [EGFR] | [EpCAM] |
| Cell line | peptides | proteins | peptides | pg/ml | pg/ml |
| Urotsa | 5465 | 763 | 0 | 147 (2) | 2.91 (0.26) |
| MGH-U3 | 10831 | 1025 | 5 | 301 (2) | 49.2 (1.0) |
| R-T4 | 7152 | 1030 | 0 | 823 (5) | 492 (12) |
| SW-780 | 4054 | 575 | 0 | 605 (9) | 26.8 (1.0) |
| RT112 | 5830 | 777 | 4 | 283 (3) | 22.6 (1.2) |
| VM-CUB-1 | 5828 | 746 | 16 | 6588 (307) | 46.4 (4.3) |
| 5637 | 4156 | 625 | 3 | 122 (2) | 2.76 (0.05) |
| T-24 | 7023 | 906 | 0 | 95 (3) | 3.27 (0.26) |
| HB-CLS-2 | 7026 | 817 | 53 | 47550 (2152) | 72.1 (7.9) |

TABLE 3
Patient data and urinary EGFR summary. The total numbers of patients in each stage group
and the numbers of males and females and number of patients with grade 1, 2 or 3 UBC are
shown. Age and urinary EGFR for each stage group are shown as median (IQR). P-values and
ROC areas are provided for each stage of UBC versus non-cancer controls. The number of
positive cases in each group is calculated using a threshold of 630 pg EGFR/mg creatinine
(mean + 2 SD of the urinary EGFR concentrations in the non-cancer group).

| | | | | | EGFR | | | No. |
|---|---|---|---|---|---|---|---|---|
| | | Male/ | Grade | | (pg/mg | | | positive |
| Stage | n | Female | (G1/G2/G3) | Age | creatinine) | p-value | ROC | cases |
| Non-cancer | 60 | 45/15 | na | 75 (66-79) | 153 (91-261) | na | na | 4 (7%) |
| pTa | 184 | 140/44 | 79/84/21 | 74 (65-81) | 167 (124-236) | 0.395 | 0.536 | 3 (2%) |
| pT1 | 130 | 112/18 | 2/37/91 | 75 (67-80) | 224 (151-353) | 0.0003 | 0.650 | 13 (10%) |
| pT2+ | 122 | 97/25 | 0/6/116 | 78 (68-83) | 317 (215-658) | 0.0001 | 0.766 | 33 (27%) |

TABLE 4
Cox multivariable analysis of prognostic indicators.

| | | | Haz | | |
|---|---|---|---|---|---|
| Variable | β | Se(β) | Ratio | P-value | HR 95% CI |
| Grade 3 | 0.982 | 0.395 | 2.670 | 0.013 | 1.231, 5.792 |
| (reference | | | | | |
| grade 1 or 2) | | | | | |
| Stage pT2+ | 1.997 | 0.306 | 7.369 | <0.001 | 4.043, 13.429 |
| (reference | | | | | |
| pTa or pT1) | | | | | |
| High EGFR | 1.062 | 0.239 | 2.891 | <0.001 | 1.809, 4.620 |
| (reference | | | | | |
| low EGFR) | | | | | |

TABLE 5
Cox multivariable analysis of prognostic
indicators including EpCAM.

| | | | Haz | | |
|---|---|---|---|---|---|
| Variable | β | Se(β) | Ratio | P-value | HR 95% CI |
| Grade 3 | 1.005 | 0.394 | 2.732 | 0.011 | 1.262, 5.912 |
| (reference | | | | | |
| grade 1 or 2) | | | | | |
| Stage pT2+ | 1.892 | 0.301 | 6.632 | <0.001 | 3.680, 11.953 |
| (reference | | | | | |
| pTa or pT1) | | | | | |
| High EGFR | 1.900 | 0.344 | 6.691 | <0.001 | 3.411, 13.124 |
| (reference | | | | | |
| low EGFR) | | | | | |
| High EpCAM | 0.756 | 0.298 | 2.131 | 0.011 | 1.189, 3.818 |
| (reference | | | | | |
| low EpCAM) | | | | | |

### REFERENCES

Adamczyk K, Tehrani M, Warnken U, Schmiegel W, Schnölzer M, Schwarte-Waldhoff I (2011) Characterization of soluble and exosomal forms of the EGFR released from pancreatic cancer cells. Life Sci 89: 304-312

Babjuk M, Oosterlinck W, Sylvester R, Kaasinen E, Böhle A, Palou-Redorta J, Rouprêt M, European Association of Urology (2011) EAU guidelines on non-muscle-invasive urothelial carcinoma of the bladder, the 2011 update. Eur Urol 59: 997-1008

Baron A, Cora E, Lafky J, Boardman C, Buenafe M, Rademaker A, Liu D, Fishman D, Podratz K, Maihle N (2003) Soluble epidermal growth factor receptor (sEGFR/sErbB1) as a potential risk, screening, and diagnostic serum biomarker of epithelial ovarian cancer. Cancer Epidemiol Biomarkers Prey 12: 103-113

Bryan R, Kirby R, O'Brien T, Mostafid H (2014a) So Much Cost, Such Little Progress. European Urology 66: 253-262

Bryan R, Shimwell N, Wei W, Devall A, Pirrie S, James N, Zeegers M, Cheng K, Martin A, Ward D (2014b) Urinary EpCAM in urothelial bladder cancer patients: characterisation and evaluation of biomarker potential. Br J Cancer 110: 679-685

Burger M, Catto J, Dalbagni G, Grossman H, Herr H, Karakiewicz P, Kassouf W, Kiemeney L, La Vecchia C, Shariat S, Lotan Y (2013) Epidemiology and risk factors of urothelial bladder cancer. Eur Urol 63: 234-241

Chow N, Liu H, Lee E, Chang C, Chan S, Cheng H, Tzai T, Lin J (1997) Significance of urinary epidermal growth factor and its receptor expression in human bladder cancer. Anticancer Res 17: 1293-1296

Hansen A, Freeman T, Arnold S, Starchenko A, Jones-Paris C, Gilger M, Washington M, Fan K, Shyr Y, Beauchamp R, Zijlstra A (2013) Elevated ALCAM shedding in colorectal cancer correlates with poor patient outcome. Cancer Res 15: 2955-2964

Huang Z, Lin L, Gao Y, Chen Y, Yan X, Xing J, Hang W (2011) Bladder cancer determination via two urinary metabolites: a biomarker pattern approach. Mol Cell Proteomics 10: 1-10

Hudelist G, Köstler W, Gschwantler-Kaulich D, Czerwenka K, Kubista E, Müller R, Helmy S, Manavi M, Zielinski C, Singer C (2006) Serum EGFR levels and efficacy of trastuzumab-based therapy in patients with metastatic breast cancer. Eur J Cancer 42: 186-192

Kandimalla R, Masius R, Beukers W, Bangma C, Orntoft T, Dyrskjot L, van Leeuwen N, Lingsma H, van Tilborg A, Zwarthoff E (2013) A 3-plex methylation assay combined with the FGFR3 mutation assay sensitively detects recurrent bladder cancer in voided urine. Clin Cancer Res 19: 4760-4769

Kaplan A, Litwin M, Chamie K The future of bladder cancer care in the USA. Nat Rev Urol 11: 59-62

Kaufman D, Shipley W, Feldman A (2009) Bladder cancer. Lancet 374: 239-249

Lemos-González Y, Rodríguez-Berrocal F, Cordero O, Gómez C, Páez de la Cadena M (2007) Alteration of the serum levels of the epidermal growth factor receptor and its ligands in patients with non-small cell lung cancer and head and neck carcinoma. Br J Cancer 96: 1569-1578

Lipponen P, Eskelinen M (1994) Expression of epidermal growth factor receptor in bladder cancer as related to established prognostic factors, oncoprotein (c-erbB-2, p53) expression and long-term prognosis. Br J Cancer 69: 1120-1125

Müller V, Witzel I, Pantel K, Krenkel S, Lück H, Neumann R, Keller T, Dittmer J, Jänicke F, Thomssen C (2006) Prognostic and predictive impact of soluble epidermal growth factor receptor (sEGFR) protein in the serum of patients treated with chemotherapy for metastatic breast cancer. Anticancer Res 26: 1479-1487

Neal D, Sharples L, Smith K, Fennelly J, Hall R, Harris A (1190) The epidermal growth factor receptor and the prognosis of bladder cancer. Cancer 65: 1619-1625

Orenes-Piñero E, Cortón M, González-Peramato P, Algaba F, Casal I, Serrano A, Sánchez-Carbayo M (2007) Searching urinary tumor markers for bladder cancer using a two-dimensional differential gel electrophoresis (2D-DIGE) approach. J Proteome Res 11: 4440-4448

Perez-Torres M, Valle B, Maihle N, Negron-Vega L, Nieves-Alicea R, Cora E (2008) Shedding of epidermal growth factor receptor is a regulated process that occurs with overexpression in malignant cells. Exp Cell Res 314: 2907-2918

Rebouissou S, Bernard-Pierrot I, de Reyniès A, Lepage M, Krucker C, Chapeaublanc E, Hérault A, Kamoun A, Caillault A, Letouzé E, Elarouci N, Neuzillet Y, MoliniéV, Vordos D, Laplanche A, Maillé P, Soyeux P, Ofualuka K, Reyal F, Biton A, Sibony M, Paoletti X, Southgate J, Benhamou S, Lebret T, Allory Y, Radvanyi F (2014) EGFR as a potential therapeutic target for a subset of muscle-invasive bladder cancers presenting a basal-like phenotype. Sci Transl Med 6: 244ra91

Shimwell N, Bryan R, Wei W, James N, Cheng K, Zeegers M, Johnson P, Martin A, Ward D (2013) Combined proteome and transcriptome analyses for the discovery of urinary biomarkers for urothelial carcinoma. Br J Cancer 108: 1854-1861

Wilken J, Perez-Torres M, Nieves-Alicea R, Cora E, Christensen T, Baron A, Maihle N (2013) Shedding of soluble epidermal growth factor receptor (sEGFR) is mediated by a metalloprotease/fibronectin/integrin axis and inhibited by cetuximab. Biochemistry 52

Witjes J, Compérat E, Cowan N, De Santis M, Gakis G, Lebret T, Ribal M, Van der Heijden A, Sherif A (2014) EAU guidelines on muscle-invasive and metastatic bladder cancer: summary of the 2013 guidelines. Eur Urol 65: 778-792

Witters L, Curley E, Kumar R, Chinchilli V, Harvey J, Crebbin V, Harvey H, Lipton A (1995) Epidermal growth factor receptor ectodomain in the urine of patients with squamous cell carcinoma. Clin Cancer Res 1: 551-557

Zampino M, Magni E, Santoro L, Zorzino L, Dell'Orto P, Sonzogni A, Fazio N, Monfardini L, Chiappa A, Biffi R, de Braud F (2008) Epidermal growth factor receptor serum (sEGFR) level may predict response in patients with EGFR-positive advanced colorectal cancer treated with gefitinib?. Cancer Chemother Pharmacol 63: 139-148

Zatovicova M, Sedlakova O, Svastova E, Ohradanova A, Ciampor F, Arribas J, Pastorek J, Pastorekova S (2005) Ectodomain shedding of the hypoxia-induced carbonic anhydrase IX is a metalloprotease-dependent process regulated by TACE/ADAM17. British Journal of Cancer 93: 1267-1276

Zeegers M, Bryan R, Langford C, Billingham L, Murray P, Deshmukh N, Hussain S, James N, Wallace D, Cheng K (2010) The West Midlands Bladder Cancer Prognosis Programme: rationale and design. BJU Int 105: 784-788

What is claimed is:

1. A method for facilitating in the prognosis of a subject having urothelial bladder cancer (UBC), the method comprising:
detecting in a sample of urine, a level of one or more fragments of Epithelial cell adhesion molecule (EpCAM) and/or epidermal growth factor receptor (EGFR) which are shed by UBC cells into the urine;
wherein the subject is determined to have a poor prognosis when a level of each or all of said one or more fragments of EpCAM and/or EGFR in the sample of urine is/are elevated and
wherein the subject is determined to have a good prognosis when a level of all of said one or more fragments of EpCAM and/or EGFR in the sample of urine is/are not elevated.

2. The method according to claim 1, wherein said fragment or fragments comprises an extracellular portion of EpCAM and/or EGFR.

3. The method according to claim 1 wherein levels of both EpCAM and EGFR are detected.

4. The method according to claim 1 wherein a level of one or more further protein fragments is also be detected, such as hepatocyte growth factor activator inhibitor type 1 (HAI-1) and/or midkine (MDK).

5. The method according to claim 1 wherein an elevated level is a level which is two standard deviations (2SD) or more above a normal or a reference value, which is typically a mean of a normal reference range as determined from a population of subjects without UBC.

6. The method according to claim 1, wherein the urine sample is subjected to process designed to isolate and/or separate said fragments from other material, such as cells or cell debris, which may be present in the urine sample.

7. The method according to claim 6 wherein the isolation/separation process comprises one or more filtration, centrifugation, mass separation, chromatography or electrophoresis steps.

8. The method according to claim 7 wherein the isolation/separation process comprises one or more filtration, or centrifugation steps.

9. The method according to claim 1 wherein said fragment or fragments are detected by immunohistochemistry, Western blot analysis, immunoblotting, ELBA, immunoprecipitation, lateral flow immunoassay, or radioimmunoassay.

10. The method according to claim 1 wherein said fragment or fragments are detected by a mass spectrometry technique, such as a matrix assisted laser desorption/ionisation mass spectrometric (MALDI-MS) or LC-MS/MS and selected reaction monitoring.

11. A method of facilitating in the determination of reatment to a subject with UBC, the method comprising:
detecting in a sample of urine, a level of one or more fragments of Epithelial cell adhesion molecule (EpCAM) and/or epidermal growth factor receptor (EGFR) which are shed by UBC cells into the urine;
wherein the subject is determined to have a poor prognosis when a level of each or all of said one or more fragments of EpCAM and/or EGFR in the sample of urine is/are elevated; and
wherein the subject is determined to have a good prognosis when a level of all of said one or more fragments of EpCAM and/or EGFR in the sample of urine is/are not elevated; and
selecting a therapy for the subject dependent upon the subject being identified as having a poor or good prognosis.

12. The method according to claim 11 further including the step of administering/conducting the selected therapy.

13. A method of administering/conducting a therapy to a subject with UBC, the method comprising:
detecting in a sample of urine, a level of one or more fragments of Epithelial cell adhesion molecule (EpCAM) and/or epidermal growth factor receptor (EGER) which are shed by UBC cells into the urine;
wherein the subject is determined to have a poor prognosis when a level of each or all of said one or more fragments of EpCAM and/or EGFR in the sample of urine is/are elevated; and
wherein the subject is determined to have a good prognosis when a level of all of said one or more fragments of EpCAM and/or EGFR in the sample of urine is/are not elevated;
selecting and administering/conducting a therapy to the subject dependent upon the subject being identified as having a poor or good prognosis.

14. The method according to claim 12 wherein the subject is identified as having a poor prognosis and the therapy is cystectomy and/or anti-EpCAM/anti-EGFR therapy.

15. An assay system for use in a method of claim 1 comprising a measurement device that measures a level of one or more fragments of Epithelial cell adhesion molecule (EpCAM) and/or epidermal growth factor receptor (EGFR) in a urine sample, in order to provide data in relation to the level of EpCAM and/or EGFR fragments in urine.

16. The system according to claim 15, further comprising a data transformation device that acquires the EpCAM and/or EGFR fragment level(s) data from the measurement device and performs data transformation to calculate whether or not the level determined is elevated or not.

17. The system according to claim 16, further comprising a user interface output device to output data to a user.

18. The system according to claim 17 further comprising a database of treatment information, wherein the device identifies treatment information in the database for the level of EpCAM and/or EGFR fragment(s) determined and outputs the treatment information to the user interface output device.

19. A kit for use in a method according to claim 1, the kit comprising at least one antibody, or probe which is/are capable of specifically binding to EpCAM and/or EGFR protein fragment(s), and may be labeled for example with a chemical, fluorescent or luminescent label and optionally instructions for use in the method.
